(19) United States
(12) Patent Application Publication
(10) Pub. No.: US 2023/0410992 A1
(43) Pub. Date: Dec. 21, 2023

(54) DATA OWNERSHIP COMPLIANCE SOLUTION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian William QUIST, Salem, NH (US); Akshay Damodar ACHARYA, Cambridge, MA (US); Naomi Beth DOHERTY, Middleton, MA (US); Escipion Rafael BAEZ, Andover, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/252,492

(22) PCT Filed: Dec. 10, 2021
(86) PCT No.: PCT/US2021/062806

§ 371 Date: May 10, 2023

Related U.S. Application Data
(60) Provisional application No. 63/123,577, filed on Dec. 10, 2020.

(51) Int. Cl. G16H 40/20; G16H 40/67

(52) U.S. Cl. CPC G16H 40/20; G16H 40/67

(57) ABSTRACT

A method if disclosed for operating a computing device communicatively coupled to a cloud-based data management system. The method includes receiving, by an application operating on the computing device, an input to initiate a patient session using a visualization system; determining, by the application, whether a compliance confirmation device is included in a computer network associated with the visualization system, wherein the compliance confirmation device is associated with an identification of a facility; responsive to determining the compliance confirmation device is included in the computer network, receiving the identification of the facility from the compliance confirmation device; obtaining, using the application, data during the patient session; receiving a request to transmit the data; transmitting the identification of the facility to the cloud-based data management system for verification; and responsive to verifying the identification of the facility, transmitting the data for storage at the cloud-based data management system.

### CROSS-REFERENCE TO RELATED APPLICATIONS

This application claims the benefit of U.S. Provisional Application Ser. No. 63/123,577 filed Dec. 10, 2020, which provisional application is incorporated by reference herein as if reproduced in full below.

### BACKGROUND

Multiple types of data may be obtained during a medical procedure, such as a surgery. The types of data may include images and/or videos obtained using a visualization system. The data may include electronic protected health information (ePHI) that is produced, saved, transferred, and/or received in electronic form from the visualization system. The production, storage, and/or transmittal of the data may comply with one or more regulations, laws (e.g., the Health Insurance Portability and Accountability Act), and/or policies. A surgeon may use a computing device to obtain the data using different visualization systems located and operated at different facilities associated with different entities.

### SUMMARY

In some examples, a method of operating a computing device communicatively coupled to a cloud-based data management system, the method comprising: receiving, by an application operating on the computing device, an input to initiate a patient session using a visualization system, wherein the computing device is communicatively coupled to the visualization system; determining, by the application, whether a compliance confirmation device is included in a computer network associated with the visualization system, wherein the compliance confirmation device is associated with an identification of a facility at which the visualization system is used; responsive to determining the compliance confirmation device is included in the computer network associated with the visualization system, receiving the identification of the facility from the compliance confirmation device; obtaining, using the application, data during the patient session; receiving a request to transmit the data to the cloud-based data management system; transmitting the identification of the facility to the cloud-based data management system for verification; and responsive to verifying the identification of the facility, transmitting the data for storage at the cloud-based data management system.

In some examples, a system includes a memory device storing instructions, and a processing device communicatively coupled to the memory device. The processing device executes the instructions to: receive, by an application implemented by the instructions, an input to initiate a patient session using a visualization system, wherein the processing device is communicatively coupled to the visualization system; determine, by the application, whether a compliance confirmation device is included in a computer network associated with the visualization system, wherein the compliance confirmation device is associated with an identification of a facility at which the visualization system is used; responsive to determining the compliance confirmation device is included in the computer network associated with the visualization system, receive the identification of the facility from the compliance confirmation device; obtain, using the application, data during the patient session; receive a request to transmit the data to a cloud-based data management system; transmit the identification of the facility to the cloud-based data management system for verification; and responsive to verifying the identification of the facility, transmit the data for storage at the cloud-based data management system.

In some examples, a tangible, non-transitory computer-readable medium stores instructions that, when executed, cause a processing device to: receive, by an application implemented by the instructions, an input to initiate a patient session using a visualization system, wherein the processing device is communicatively coupled to the visualization system; determine, by the application, whether a compliance confirmation device is included in a computer network associated with the visualization system, wherein the compliance confirmation device is associated with an identification of a facility at which the visualization system is used; responsive to determining the compliance confirmation device is included in the computer network associated with the visualization system, receive the identification of the facility from the compliance confirmation device; obtain, using the application, data during the patient session; receive a request to transmit the data to a cloud-based data management system; transmit the identification of the facility to the cloud-based data management system for verification; and responsive to verifying the identification of the facility, transmit the data for storage at the cloud-based data management system.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the femur from below and looking into the intercondylar notch;

FIG. 4 shows a surgical system in accordance with at least some embodiments;

FIG. 5 shows a block diagram of a use case of using a compliance confirmation device at different facilities in accordance with at least some embodiments;

FIG. 6 shows flow diagrams for setting up a compliance confirmation device and on-going procedures of a cloud-based data management system in accordance with at least some embodiments;

FIG. 7 is an example method for using a compliance confirmation device to enable uploading data obtained by an application of a user in accordance with at least some embodiments;

FIG. 8 is an example method for displaying various information pertaining at least to a compliance confirmation device in accordance with at least some embodiments;

FIG. 9 is an example method for error handling when attempting to upload data to a cloud-based data management system in accordance with at least some embodiments; and

FIG. 10 shows a computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

The terms “input” and “output” when used as nouns refer to connections (e.g., electrical, software), and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections. In systems implemented in software, these “inputs” and “outputs” define parameters read by or written by, respectively, the instructions implementing the function.

“Controller” shall mean, alone or in combination, individual circuit components, an application specific integrated circuit (ASIC), a microcontroller with controlling software, a reduced-instruction-set computing (RISC) with controlling software, a digital signal processor (DSP), a processor with controlling software, a programmable logic device (PLD), or a field programmable gate array (FPGA), configured to read inputs and drive outputs responsive to the inputs.

“Real-time” may refer to an action, operation, method, function, instruction, etc. being performed within 2 seconds. “Near real-time” may refer to an action, operation, method, function, instruction, etc. being performed between 2 seconds and 1 minute.

### DETAILED DESCRIPTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Users or surgeons often use a visualization system and/or a computing device (e.g., tablets) to obtain data (e.g., images, patient notes, and/or video) of portions of a patient's body during a patient session (e.g., surgery). The surgeons may share the images taken with the patient post operation and/or view the images after the operation to determine a diagnosis. It may be desirable to store the data at a central cloud storage repository, such as a cloud-based data management system, such that the surgeon is able to access the data at one secure location. The data may include electronic protected health information (ePHI) that is produced, saved, transferred, and/or received in electronic form from the visualization system. The production, storage, and/or transmittal of the data may comply with one or more regulations, laws (e.g., the Health Insurance Portability and Accountability Act (HIPPA)), and/or policies.

Conventionally, computing devices used to upload the data may be fixed within the facilities, which may ensure compliance with data ownership and storage for HIPPA and/or other suitable regulation. For example, an entity that manages and operates the cloud-based data manage system may require each entity to execute a contractual agreement (e.g., Business Associates Agreement) that permits the entity to store ePHI data in the cloud-based data management system.

A technical problem may arise when users or surgeons work across multiple facilities associated with different entities or organizations. For example, the surgeon may be a valid employee at one or more hospitals, urgent care facilities, private practice, or the like. The different entities may be unaffiliated with each other. Moreover, an application (e.g., software application) installed on the computing device used by the surgeon may use a cloud account linked to a first facility but not linked to a second facility. The second facility may not have a signed contractual agreement with the entity managing the cloud-based data management system 442. However, the surgeon may use the computing device at the second facility to attempt to upload data from the second facility using the cloud account linked to the first facility, which may be undesirable.

Another problem may arise when a camera control unit of a visualization system is swapped or replaced at a facility. This may occur for a number of reasons including repairs, updating software, etc. Thus, linking the facility to a specific camera control unit would quickly be out of date and not solve the problem.

A technical solution may include using a compliance confirmation device associated with each visualization system to ensure that the surgeons can only upload data obtained from the facilities that have executed contractual agreements with the entity managing the cloud-based management system. For example, the compliance confirmation device may be added to a private area network with the visualization system. The compliance confirmation device may provide an identification of a facility when queried by an application executing on the computing device of the surgeon. The identification of the facility may provide a digital indication that the entity associated with the facility has executed the appropriate contractual agreement with the entity associated with the cloud-based data management system. The use of the compliance confirmation device may enable the rapid replacement and/or swapping of other equipment (e.g., camera control unit, tablet, etc.) without compromising the data ownership and/or management compliance of a facility.

Each facility may use a mechanism to indicate a connected visualization system is approved for uploads to the cloud-based data management system. The mechanism may include the compliance confirmation device that is shipped to the facility when the contractual agreement between the entity associated with the facility and the entity associated with the cloud-based data management system is executed. In some embodiments, the compliance confirmation device may be added to a private area network to which a computing device (e.g. tablet) of a surgeon and/or the visualization system is also connected. The compliance confirmation device may be configured to provide an identification of a facility to an application executing on the computing device when data uploads are attempted.

In some embodiments, the compliance confirmation device may refer to a virtual compliance confirmation device that is installed within a controller of the visualization system. The virtual compliance confirmation device may reside between a firewall or restricted area of an operating system operating on the controller. The virtual compliance confirmation device may be installed and configured after the contractual agreement is executed. The virtual compliance confirmation device may be configured to provide the identification of the facility to the application executing on the computing device.

A surgeon may use the visualization system to obtain data during a patient session. The data may be tagged or associated with the identification of the facility and stored in memory of the computing device. A patient session may be saved and closed upon completion of a procedure. The application may switch from operating in a computer network connected mode (e.g., using the private area network) to an internet-based protocol (e.g., Wi-Fi) mode to connect to the cloud-based management system.

When an upload is triggered (e.g., either manually or electronically), the identification of the facility and license credentials of the facility and/or surgeons may be transmitted to the cloud-based data management system. The cloud-based data management system may determine whether the license credentials are valid and the identification of the facility associated with the data is valid. If both are approved as valid, the cloud-based data management system may initiate a cloud session to enable the data to be uploaded and stored at the cloud-based data management system. If the license credentials are incorrect and/or the identification of the facility is incorrect for the data of the patient, the cloud-based data management system may notify the application. The application may present an error message indicating the identification of the facility associated with the data of the patient is incorrect and/or the license credentials are incorrect. An administrator and/or the surgeon may be enabled to correct the identification of the facility.

In some embodiments, the present disclosure enables an entity associated with a facility and/or surgeon precise control over uploading data obtained from a visualization system. Specific visualization systems may be selected for uploading of data. Further, the visualization systems may be programmed such that certain surgeons or subsets of surgeons can upload data from certain visualization systems. Some advantages of the disclosed techniques may enable data portability while maintaining proper lines of data ownership with minimal setup and/or maintenance.

The various examples were developed in the context of ACL repair, and thus the discussion below is based on the developmental context. However, the techniques are applicable to many types of ligament repair, such as medial collateral ligament repair, lateral collateral ligament repair, and posterior cruciate ligament repair. Moreover, the various example methods and systems can also be used for planning and placing anchors to reattach soft tissue, such as reattaching the labrum of the hip, the shoulder, or the meniscal root. Thus, the description and developmental context shall not be read as a limitation of the applicability of the teachings. In order to orient the reader, the specification first turns a description of the knee.

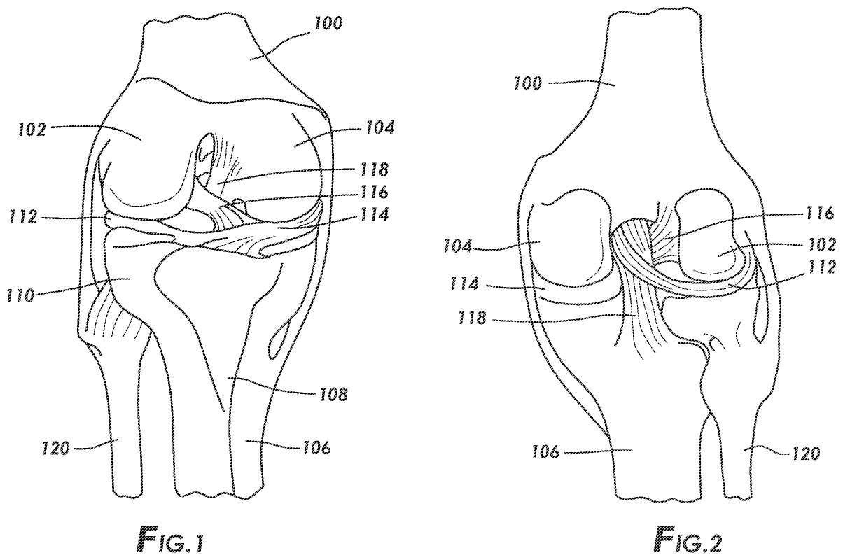

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy's tubercle 110. Disposed between the femoral condyles 102 and 104 and the tibia 106 are the lateral meniscus 112 and the medial meniscus 114. Several ligaments are also visible in the view of FIG. 1, such as the ACL 116 extending from the lateral side of femoral notch to the medial side of the tibia 106. Oppositely, the posterior cruciate ligament 118 extends from medial side of the femoral notch to the tibia 106. Also visible is the fibula 120, and several additional ligaments that are not specifically numbered.

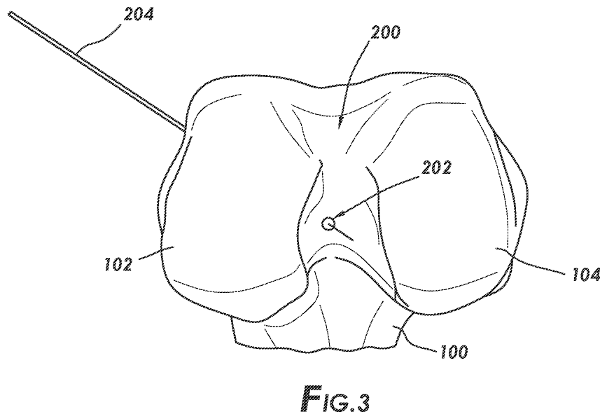

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the tunnel entry point for the graft is at the anatomical attachment location of the native ACL. Such tunnel placement at the attachment location of the native ACL attempts to recreate original knee kinematics. In arthroscopic surgery, the location of the tunnel through the tibia 106 is relatively easy to reach, particularly when the knee is bent or in flexion. However, the tunnel through the femur 100 resides within the intercondylar notch. Depending upon the physical size of the patient and the surgeon's selection for location of the port through the skin, and through which the various instruments are inserted into the knee, it may be difficult to reach the attachment location of the native ACL to the femur 100.

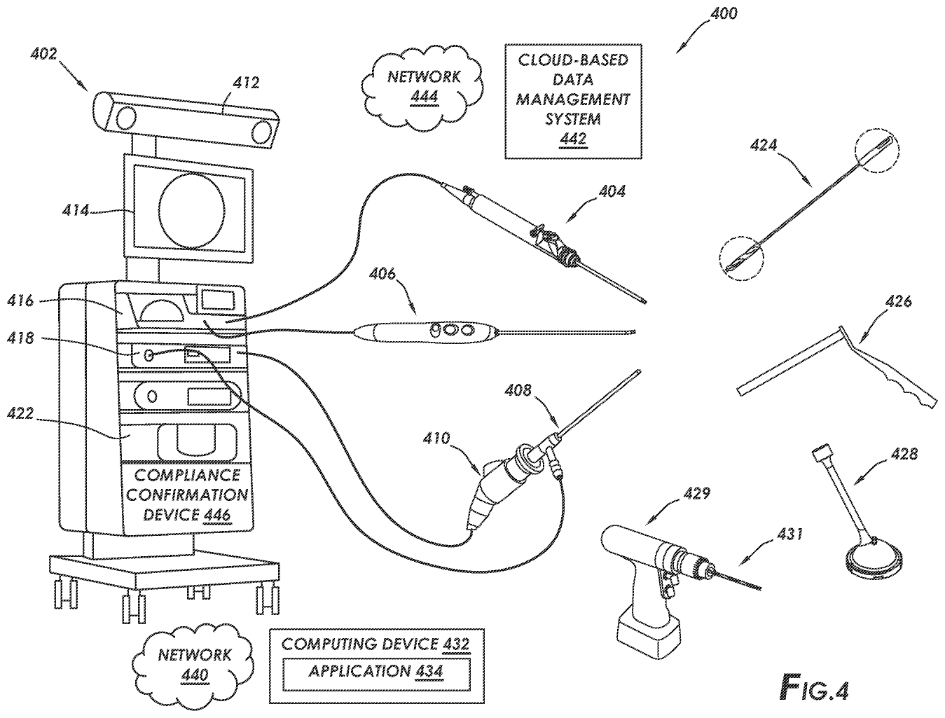

FIG. 3 shows a view of the femur from below and looking into the intercondylar notch. In particular, visible in FIG. 3 are the lateral condyle 102 and the medial condyle 104. Defined between the femoral condyles 102 and 104 is the femoral notch 200. The femoral tunnel may define inside aperture 202 within the femoral notch 200, the inside aperture 202 closer to the lateral condyle 102 and displaced into the posterior portion of the femoral notch 200. The femoral tunnel extends through the femur 100 and forms an outside aperture on the outside or lateral surface of the femur 100 (the outside aperture not visible in FIG. 3). FIG. 3 shows an example drill wire 204 that may be used to create an initial tunnel or pilot hole. Once the surgeon verifies that the drill wire 204 is closely aligned with a planned-tunnel path, the femoral tunnel is created by boring or reaming with another instrument (e.g., a reamer) that may use the drill wire 204 as a guide.

Drilling of a tunnel may take place from either direction. Considering the femoral tunnel again as an example, the tunnel may be drilled from the outside or lateral portion of the femur 100 toward and into the femoral notch 200, which is referred to as an “outside-in” procedure. Oppositely, the example femoral tunnel may be drilled from the inside of the femoral notch 200 toward and to the lateral portion of the femur 100, which is referred as an “inside-out” procedure. The various examples discussed below are equally applicable to outside-in or inside-out procedures. Outside-in procedures may additionally use a device which holds the drill wire on the outside portion, and physically shows the expected tunnel location of the inside aperture within the knee. However, the device for the outside-in procedure is difficult to use in arthroscopic procedures, and thus many arthroscopic repairs use the inside-out procedure. The further examples discussed below are thus based on an inside-out procedure, but such should not be read as a limitation. The specification now turns to an example surgical system.

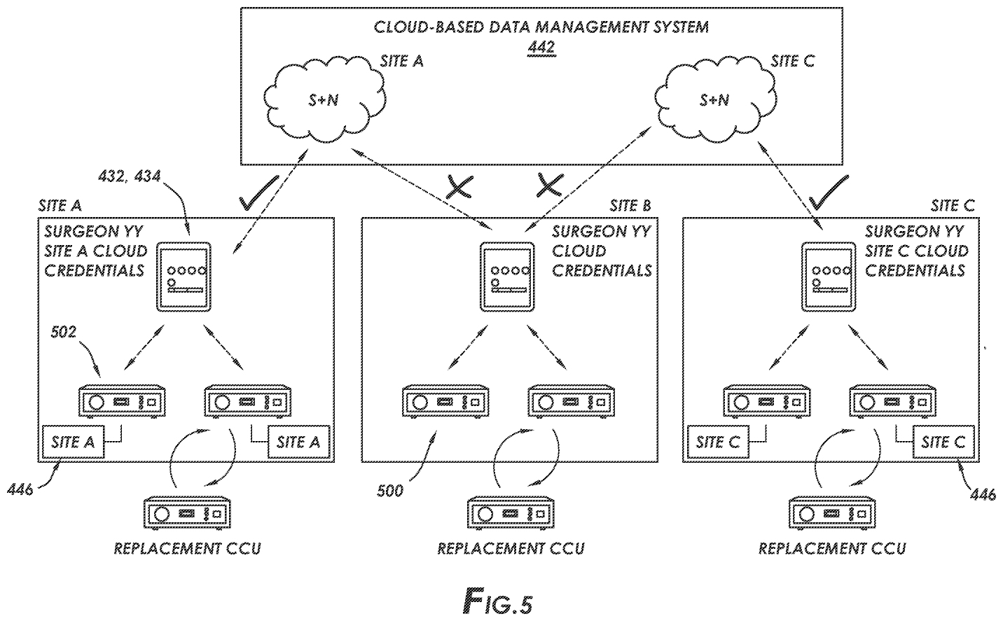

FIG. 4 shows a surgical system (not to scale) or a visualization system in accordance with at least some embodiments. In particular, the example surgical system or visualization system 400 comprises a tower or device cart 402, an example mechanical resection instrument 404, an example plasma-based ablation instrument (hereafter just ablation instrument 406), and an endoscope in the example form of an arthroscope 408 and attached camera head 410. The device cart 402 may comprise a camera 412 (illustratively shown as a stereoscopic camera), a display device 414, a resection controller 416, and a camera control unit (CCU) together with an endoscopic light source and video controller 418. In example cases the CCU and video controller 418 not only provides light to the arthroscope 408 and displays images received from the camera head 410, but implements various additional aspects, such as determining values of one or more parameters associated with a reamer (e.g., actual depth of reamer within a bone, diameter of reamer, identifier of reamer, etc.), displaying values of the one or more parameters on the display device 414, receiving input to modify an operating parameter (e.g., revolutions per minute, power on/off, drilling direction, etc.) of a drill associated with the reamer, controlling operation of a drill based on the one or more parameters, and so forth. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The surgical controller 418 may also transmit data (e.g., images and/or video) obtained during a procedure to a computing device 432 (e.g., tablet, smartphone, laptop etc.) via a network 440. The network 440 may be a private area network that uses private address space for Internet Protocol addresses. In some embodiments, the network 440 may be a personal area network or a local area network. The network 440 may be used by one or more devices of the device cart 402, a compliance confirmation device 446, and the computing device 432 during a patient session where a surgeon is performing a surgery. The compliance confirmation device 446 may include a processing device, a memory device, a network interface device, or any suitable component to perform the techniques described herein. For example, the compliance confirmation device 446 may be capable of connecting to the network 440 and communicating information to other devices connected to the network 440.

The computing device 432 may execute an application 434 implemented in computer instructions stored on one or more memory devices and executed by one or more processing devices. The application may query the network 440 to determine which devices are connected to the network 440. Additionally or alternatively, a compliance confirmation device 446 may be added to the network 440 and emit a heartbeat signal to other devices connected to the network 440.

Upon determining the compliance confirmation device 446 is included on the network 440, the application 434 may receive the identification of the facility in which the visualization system is being used. The identification may be a device type, a stock keeping unit, or any suitable unique identifier. The application 434 may provide a user interface that displays a graphical element that indicates the compliance confirmation device 446 is included in the network 440. Such a graphical element provides an indication to the surgeon that the contractual agreement has been properly executed between the entity associated with the facility and the entity managing 1 cloud-based data management system 442 that stores the patient's data. The contractual agreement may include terms that identify the entity associated with the facility as the owner of the data uploaded from the facility, and terms that specify the data is securely stored and HIPPA compliant by the cloud-based data management 442.

The cloud-based data management system 442 may include one or more servers in connected in a distributed network environment. The one or more servers may host one or more databases to store data received from the application 434. The cloud-based management system 442 may generate accounts for the facilities and licenses including license identifiers and/or license credentials for the accounts. The surgeons that are associated with a respective facility may receive a respective license (e.g., identifier and/or credentials) when the contractual agreement is executed between the entity associated with the facility and the entity associated with the cloud-based data management system 442.

The cloud-based data management system 442 may maintain a list of identifications of facilities that are approved to upload data. In some embodiments, a customer service representative may control this list. The cloud-based data management system 442 may maintain a mapping of an identification of a facility to an identification (e.g., serial number) of a CCU used by a visualization system. In some examples, the identification of the CCU may only be linked to a single identification of the facility. The cloud-based data management system 442 may be configured to enable mapping the identification of the facility to the identification of the CCU. For example, in some embodiments, a representative of the entity associated with the cloud-based data management system 442 may login to a representative portal using a computing device (e.g., smartphone) and search for a surgeon and/or facility. The representative may be enabled, using the representative portal, to manage linked CCUs by adding a CCU, removing a CCU, etc. In some examples, a serial number of the CCU may not be used to manage the linked CCUs in the portal.

In some embodiments, the representative portal may be configured to program a CCU with an identification of a facility. For example, a settings page may enable a representative to add a character string representing the identification of the facility to the CCU. The setting may be password protected. The application 434 may query the CCU for the identification of the facility and append the identification to data obtained by the application intra-operatively.

The application 434 may enter a setup mode to configure the compliance confirmation device 446. The application 434 may enter several data fields that are saved in the configuration of the compliance confirmation device 446. For example, the data fields may include an identifier of the network 440, a password for the network 440, the identification of the facility, a license identifier, a license password, a compliance contract identifier, a cloud-based data management system entity identifier, a user cloud account login identifier, a user cloud account password, and/or a generic identifier that is alphanumeric. The application 434 may receive a serial number of the CCU used by the visualization system. The application 434 may be logged into with a single license associated with the facility and the cloud-based data management system 442. The application 434 may display the identification of the facility and that the facility is associated with the license. The application 434 may be configured to transmit a list of CCUs from which data was captured and received if the CCU is approved for uploading of data to the cloud-based data management system 442.

When the application 434 obtains data from the visualization system, the application 434 may tag or associate each file in the data with the identification of the facility (received from the compliance confirmation device 446) and/or a ship-to-customer number. The application 434 may store each file in a memory device of the computing device 432. At the time of upload, the application 434 may transmit the identification of the facility associated with each file and the license information (e.g., identifier and/or credentials) to the cloud-based data management system 442 for verification. If verified, a cloud session may be initiated and the data may be uploaded to the cloud-based data management system 442. If not, the upload may be blocked.

In some embodiments, the application 434 may obtain the data from the visualization system and store the data in a memory device of the computing device 432. The application 434 may trigger an upload to the cloud-based data management system 442. At the time of upload, the application 434 may query for the presence of the compliance computing device 446 on the network 440. If the compliance computing device 446 is on the network, then the upload may be allowed. If not, the upload may be blocked.

In some embodiments, if the compliance confirmation device 446 is present on the network 440 when a patient account is created, the identification of the facility received from the compliance confirmation device 446 is associated with the data of the patient for each file obtained related to the patient.

Prior to uploading the data to the cloud-based data management system 442, the application 434 may switch from being connected to the network 440 (e.g., a private area network) to being connected to network 444. The switch may occur when the patient session has been closed. The network 444 may be a wide area network (WAN), such as the Internet, and may use an internet-based protocol (e.g., Wi-Fi) to communicate data between devices connected to the network 444. If the upload is approved, the data may be sent via the network 444 from the application 434 to the cloud-based management system 442.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 424, and another magnified portion to show a connector for coupling to chuck of a drill. Once the surgeon drills the pilot tunnel, the surgeon and/or the surgical controller 418 may then assess whether the pilot tunnel matches or closes matches the planned-tunnel path. If the pilot tunnel is deemed sufficient, then the drill wire 424 may be used as a guide for creating the full-diameter throughbore for the tunnel, or alternatively, for creating a counterbore stemming from the intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As will be discussed in greater detail below, the calibration assembly 428 may be used to detect optical distortion in images received by the surgical controller 418 through the arthroscope 408 and attached camera head 410.

Additional tools and instruments will be present, such as a drill 429 for drilling with the drill wire 424, various reamers 431 for creating the throughbore and counterbore aspects of the tunnel, and various tools for suturing and anchoring the graft in place. Some of these additional tools and instruments are not shown so as not to further complicate the figure.

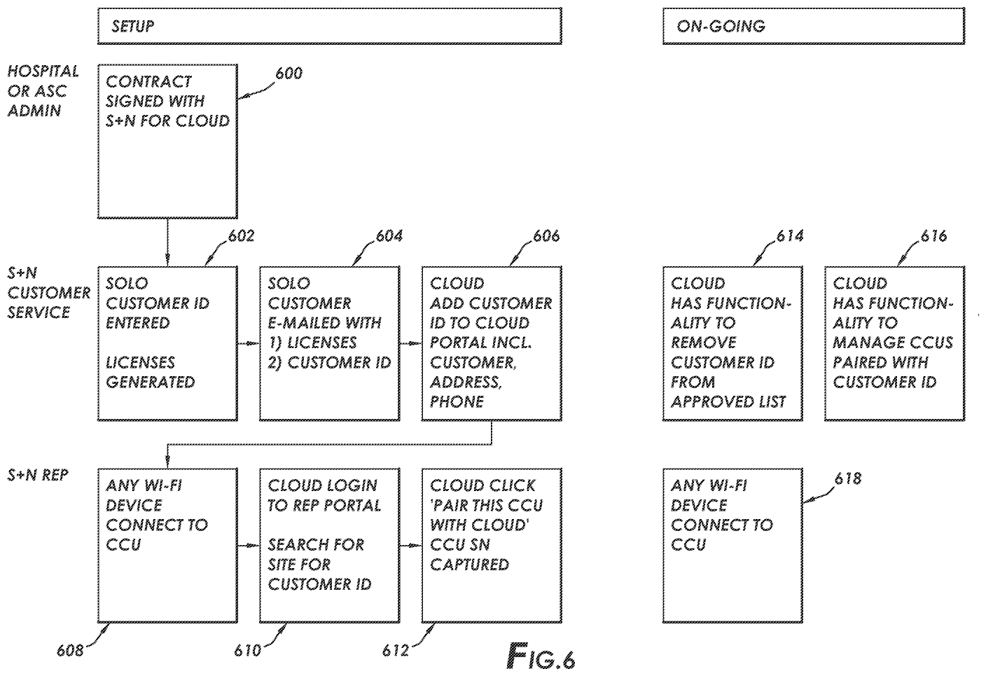

FIG. 5 shows a block diagram of a use case of using a compliance confirmation device 446 at different facilities in accordance with at least some embodiments. As depicted, Site A (also referred to as “facility A”) includes two visualization systems including two CCUs 502. An entity associated with the facility A has executed a contractual agreement with an entity associated with the cloud-based data management system 442 hosting a cloud session for “Site A”. Accordingly, two compliance confirmation devices 446 are shipped and/or installed with the two CCUs 502 on the network 440 used by the CCU and the computing device 432 at facility A. Further, Site B (also referred to as “facility B”) includes two visualization system including two CCUs 502. However, an entity associated with facility B has not executed a contractual agreement with an entity associated with the cloud-based data management system 442 (that is why there is no cloud session for facility B depicted in FIG. 5). An entity associated with the facility C has executed a contractual agreement with an entity associated with the cloud-based data management system 442 hosting a cloud session for “Site C”. Accordingly, two compliance confirmation devices 446 are shipped and/or installed with the two CCUs 502 on the network 440 used by the CCU 502 and the computing device 432 at facility C.

The surgeon YY may be employed by all three facilities A, B, and C. The surgeon may perform a patient session at facility A using one of the two CCUs 502 and the application on the computing device 432. The surgeon YY may use the application 434 and the CCU 502 to obtain data (e.g., images and/or video of the patient). The surgeon YY may request to upload the data to the cloud-based data management system 442. The application 434 may use the identification of the facility and license credentials to upload data (as represented by a check mark) to the cloud session for facility A hosted by the cloud-based data management system 442.

The surgeon YY may take the computing device 432 executing the application 434 to facility B to perform another patient session. Since facility B has not executed a contractual agreement, neither of the CCUs 502 are associated with a compliance confirmation device (as depicted by space 500). Accordingly, facility B does not have a license with the cloud-based data management system associated with an identification of a facility to use to upload data to either cloud session for facility A or facility B (as represented by “X”).

The surgeon YY may take the computing device 432 executing the application 434 to facility C to perform another patient session. The surgeon YY may use the application 434 and the CCU 502 to obtain data (e.g., images and/or video of the patient). The surgeon YY may request to upload the data to the cloud-based data management system 442. The application 434 may use the identification of the facility and license credentials to upload data (as represented by a check mark) to the cloud session for facility A hosted by the cloud-based data management system 442.

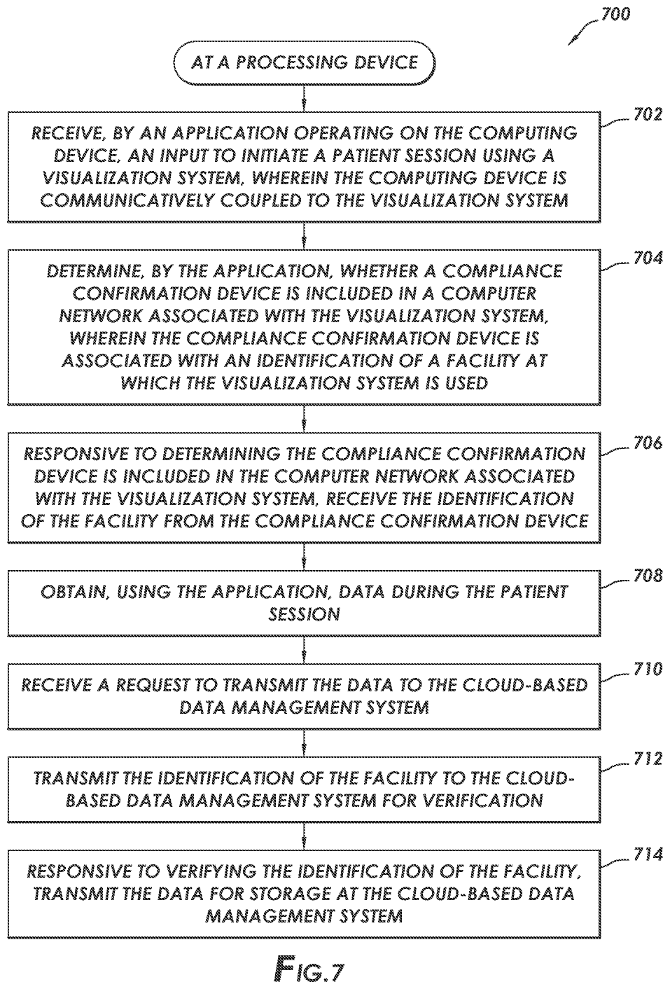

FIG. 6 shows flow diagrams for setting up a compliance confirmation device 446 and on-going procedures of a cloud-based data management system 442 in accordance with at least some embodiments. Beginning with the setup procedures, at block 600, an entity (e.g., administrator) associated with a facility (e.g., hospital) may execute a contractual agreement with an entity associated with the cloud-based data management system 442. At block 602, a customer service agent may enter an identification of the facility (e.g., also referred to as “SOLO Customer ID”), and one or more licenses (e.g., identifiers and passwords) for the facility may be generated. At block 604, the entity associated with the facility may be messaged (e.g., emailed) with the licenses information and the identification of the facility. At block 606, the cloud-based data management system 442 may add the identification of the facility, as well as a customer name, address, and/or phone number. At block 608, a representative of the entity associated with the cloud-based data management system 442 may use an internet enabled computing device (e.g., tablet, smartphone) to connect to a visualization system (e.g., CCU) and login to a representative portal associated with the cloud-based data management system 442. The representative may search for the facility using the identification of the facility, at block 610. The portal may be used to select to pair the CCU with the cloud-based data management system 442, and the serial number of the CCU may be obtained and sent to the cloud-based data management system 442 to be associated with the identification of the facility.

Turning to the on-going procedures, at block 614, the cloud-based data management system 442 may be configured to remove the identification of the facility from an approved list, to manage CCUs paired with the identification of the facility, and/or to un-pair a particular CCU from the cloud-based data management system 442.

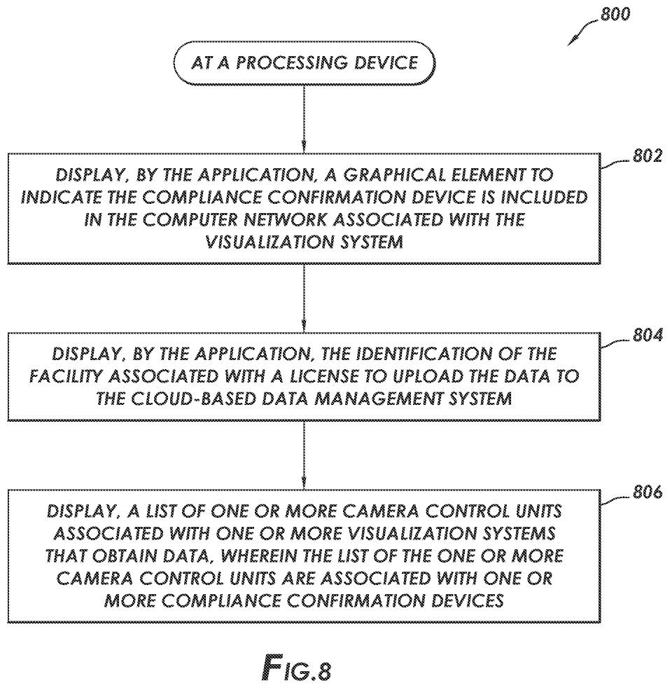

FIG. 7 is an example method 700 for using a compliance confirmation device to enable uploading data obtained by an application of a user in accordance with at least some embodiments. The method 700 may include operating a computing device communicatively coupled to a cloud-based data management system. In particular, the methods starts at block 700. The method may be performed by one or more processors, such as included in the computing device 432 (e.g., tablet of the surgeon), for example. The method may be implemented in computer instructions stored in one or more memories that are executed by the one or more processors.

At block 702, the processor of the computing device 432 may execute an application 434 implemented in computer instructions and stored in one or more memory devices (e.g., on the computing device 432, in the cloud-based data management system 442, on the surgical controller 418, etc.). The application 434 may receive an input to initiate a patient session using a visualization system. The input may be entered by a surgeon using an input peripheral (e.g., mouse, keyboard, microphone, touchscreen, etc.) of the computing device 432. In some embodiments, the computing device 432 may be communicatively coupled to the visualization system. The visualization system may be an arthroscopic tower and the compliance confirmation device may be communicatively coupled to the arthroscopic tower.

At block 704, the processor executing the application 434 may determine whether a compliance confirmation device 446 is included in a computer network 4440 associated with the visualization system. The compliance confirmation device 446 may be associated with an identification (e.g., alphanumeric string, unique identifier, etc.) of a facility at which the visualization system is used. The application 434 may determine whether the compliance confirmation device 446 is included in the computer network 440 associated with the visualization system by identifying at least one of a stock keeping unit associated with the compliance confirmation device 446 on the computer network 440, a serial number associated with the compliance confirmation device 446 on the computer network 440, and a device type associated with the compliance confirmation device 446 on the computer network 440.

An entity, such as Smith & Nephew of Andover, Massachusetts, USA, may enter into a contractual agreement with a facility that permits the entity to store ePHI data associated with users in the cloud-based data management system 442. The contractual agreement may indicate that the facility is the owner of the data to be uploaded by surgeons that perform operations at the facility. Each surgeon that works at the facility may be issued a user account to access the cloud-based data management system 442. The facility may not receive the compliance confirmation device 446 until the contractual agreement is executed between the entity and the facility. The presence of the compliance confirmation device 446 on the computer network 440 with the visualization system may ensure that the facility has signed the contractual agreement and is enabled to upload data to the cloud-based data management system 442.

At block 706, responsive to determining the compliance confirmation device 446 is included in the computer network 440 associated with the visualization system, the processor may receive the identification of the facility from the compliance confirmation device 446. In some embodiments, the application 434 may determine that the compliance confirmation device 446 is not included in the computer network 440 associated with the visualization system. In such an example, the application 434 may block the data from being transmitted to the cloud-based data management system 442.

At block 708, the processor executing the application 434 may obtain data during the patient session. For example, the data may include images and/or video of a surgery performed during the patient session. The data may be tagged or otherwise associated with the identification of the facility by the application 434 when the data is obtained and written to memory. The application 434 may associated a ship-to-customer number as a data field associated with each file including data. When the medical procedure is complete, the surgeon may select to save the patient session and close the patient session.

The processor may switch the application 434 to operate in an internet-based communication protocol (e.g., Wi-Fi) transmission mode via a network 444. In some embodiments, the internet-based communication protocol is not used by the computer network 440 communicatively connecting the visualization system, the computing device 432 (e.g., tablet), and/or the compliance confirmation device 446 during the patient session. The computer network 440 may implement a private network between the visualization system, the computing device 432, and/or the compliance confirmation device 446.

At block 710, the processor may receive a request to transmit the data to the cloud-based data management system 442. The request to transmit the data may be triggered manually by the surgeon selecting an operation on the computing device 432 and/or electronically based on one or more criteria. For example, the application 434 may use a schedule to aggregate and transmit batched data (e.g., images and video) at a certain time each day, the application 434 may determine a threshold size of data has been satisfied and transmit the data, the application 434 may periodically transmit the data to the cloud-based data management system 442, or the like.

At block 712, the processor may transmit, to the cloud-based data management system 442 for verification, the identification of the facility and the license credentials (license identifier and/or license password) associated with the account of the facility and/or the surgeon. The cloud-based data management system 442 may verify the identification of the facility with the license credentials received from the application 434. Once verified, the received license credentials may be used by the cloud-based data management system 442 to create a cloud session to be used for uploading data by the user of the application 434. The cloud session may refer to a secure licensing connection where communications between the application and the cloud-based data management system 442 may be encrypted. The secure licensing connection may be implemented using a virtual private network (VPN) and/or a dedicated private connection.

At block 714, responsive to verifying at least the identification of the facility and/or the license credentials, the processor may transmit the data for storage at the cloud-based data management system 442.

In some embodiments, the compliance confirmation device 446 may be added to or registered with the computer network 440 after a determination is made that the contractual agreement has been executed by an entity associated with the facility. Executing the contractual agreement may cause the cloud-based data management system 442 to create an account associated with the facility and one or more licenses (e.g., to be issued to the users) for the account. The licenses may be associated with credentials (e.g., user names and passwords). The application may receive at least one of the identification of the facility, a license identifier, and/or a license password.

In some examples, the application 434 may enter a setup mode to configure the compliance confirmation device 446 with the at least one of the identification of the facility, the license identifier, and/or the license password. When the compliance confirmation device 446 is active on the computer network 440, a graphical element (e.g., cloud icon, image, graphic, etc.) may be displayed by the application to indicate the compliance confirmation device is included in the computer network associated with the visualization system.

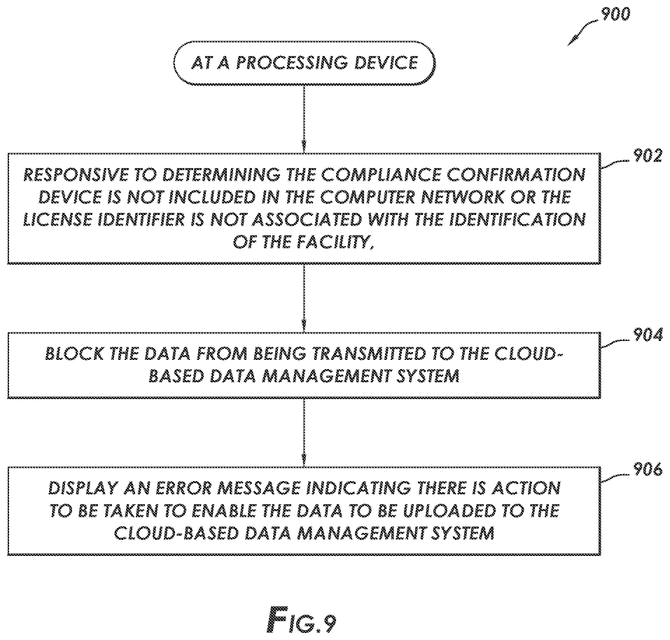

FIG. 8 is an example method 800 for displaying various information pertaining at least to a compliance confirmation device 446 in accordance with at least some embodiments. The method 800 may include operating a computing device 432 communicatively coupled to a cloud-based data management system 442. In particular, the methods starts at block 800. The method 800 may be performed by one or more processors, such as included in the computing device 432 (e.g., tablet of the surgeon), for example. The method 800 may be implemented in computer instructions stored in one or more memories that are executed by the one or more processors.

At block 802, the processor of the computing device 432 may execute an application 434 to display the identification of the facility associated with the license to upload the data to the cloud-based data management system 442. The application 434 may also display a list of one or more camera control units associated with one or more visualization systems that obtain data. The list of the one or more camera control units may be approved to upload data to the cloud-based data management system.

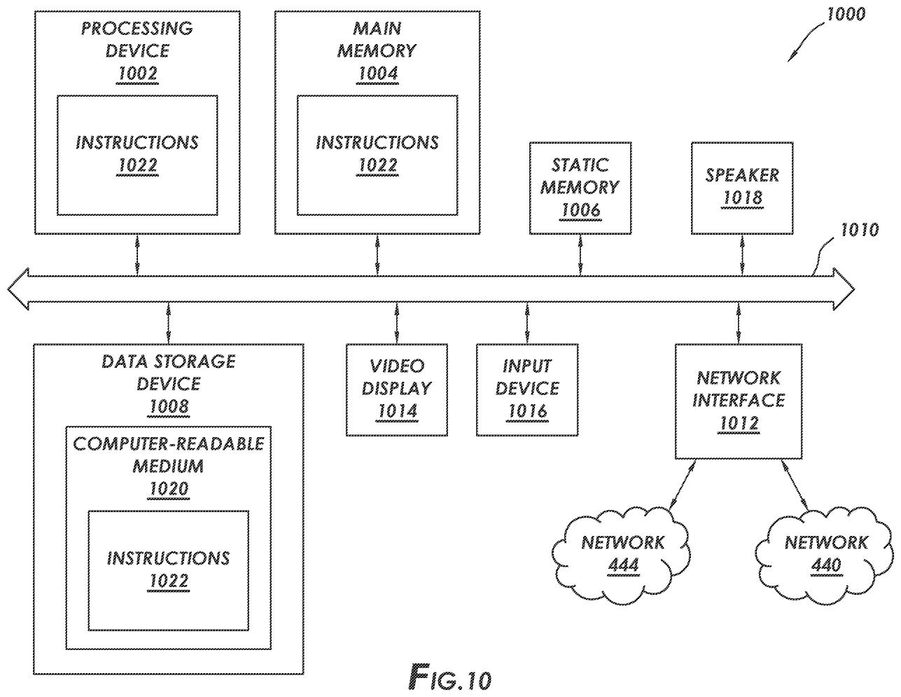

FIG. 9 is an example method 900 for error handling when attempting to upload data to a cloud-based data management system 442 in accordance with at least some embodiments. The method 900 may include operating a computing device 432 communicatively coupled to a cloud-based data management system 442. In particular, the methods starts at block 900. The method 900 may be performed by one or more processors, such as included in the computing device 432 (e.g., tablet of the surgeon), for example. The method 900 may be implemented in computer instructions stored in one or more memories that are executed by the one or more processors.

At block 902, responsive to determining the compliance confirmation device 446 is not included in the computer network 440 or the license identifier and/or password is not associated with the identification of the facility, the processor may (at block 904) block the data from being transmitted to the cloud-based data management system 442 and/or display an error message indicating there is action to be taken to enable the data to be uploaded to the cloud-based data management system 442. The action may be performed by a representative associated with the cloud-based data management system 442. For example, the representative may modify the identification of a facility that is associated with certain data for a patient that is uploaded by particular surgeon.

FIG. 10 shows an example computer system 1000. In one example, computer system 1000 may correspond to the surgical controller 418, the compliance confirmation device 446, the computing device 432 (e.g., a tablet device) within the surgical room, cloud-based data management system 442, or any other system that implements any or all the various methods discussed in this specification. The computer system 1000 may be connected (e.g., networked) to other computer systems in a local-area network (LAN), a private area network (e.g., network 440), an intranet, and/or an extranet (e.g., device cart 402 network), or at certain times the Internet (e.g., when not in use in a surgical procedure). For example, the computer system 1000 may be communicatively connected to the cloud-based data management system 442 via an internet-based protocol (e.g., Wi-Fi), such as network 444. The computer system 1000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1000 includes a processing device 1002, a main memory 1004 (e.g., read-only memory (ROM), flash memory, dynamic random access memory (DRAM) such as synchronous DRAM (SDRAM)), a static memory 1006 (e.g., flash memory, static random access memory (SRAM)), and a data storage device 1008, which communicate with each other via a bus 1010.

Processing device 1002 represents one or more general-purpose processing devices such as a microprocessor, central processing unit, or the like. More particularly, the processing device 1002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1002 may also be one or more special-purpose processing devices such as an application specific integrated circuit (ASIC), a field programmable gate array (FPGA), a digital signal processor (DSP), network processor, or the like. The processing device 1002 is configured to execute instructions for performing any of the operations and steps discussed herein. Once programmed with specific instructions, the processing device 1002, and thus the entire computer system 1000, becomes a special-purpose device, such as the surgical controller 418.

The computer system 1000 may further include a network interface device 1012 for communicating with any suitable network (e.g., the device cart 402 network). The computer system 1000 also may include a video display 1014 (e.g., display device 414), one or more input devices 1016 (e.g., a microphone, a keyboard, and/or a mouse), and one or more speakers 1018. In one illustrative example, the video display 1014 and the input device(s) 1016 may be combined into a single component or device (e.g., an LCD touch screen).

The data storage device 1008 may include a computer-readable storage medium 1020 on which the instructions 1022 (e.g., implementing any methods and any functions performed by any device and/or component depicted described herein) embodying any one or more of the methodologies or functions described herein is stored. The instructions 1022 may also reside, completely or at least partially, within the main memory 1004 and/or within the processing device 1002 during execution thereof by the computer system 1000. As such, the main memory 1004 and the processing device 1002 also constitute computer-readable media. In certain cases, the instructions 1022 may further be transmitted or received over a network via the network interface device 1012.

While the computer-readable storage medium 1020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

### CLAUSES

1. A method of operating a computing device communicatively coupled to a cloud-based data management system, the method comprising:
receiving, by an application operating on the computing device, an input to initiate a patient session using a visualization system, wherein the computing device is communicatively coupled to the visualization system;
determining, by the application, whether a compliance confirmation device is included in a computer network associated with the visualization system, wherein the compliance confirmation device is associated with an identification of a facility at which the visualization system is used;
responsive to determining the compliance confirmation device is included in the computer network associated with the visualization system, receiving the identification of the facility from the compliance confirmation device;
obtaining, using the application, data during the patient session;
receiving a request to transmit the data to the cloud-based data management system;
transmitting the identification of the facility to the cloud-based data management system for verification; and
responsive to verifying the identification of the facility, transmitting the data for storage at the cloud-based data management system.
2. The method of any clause herein, wherein determining, by the application, whether the compliance confirmation device is included in the computer network associated with the visualization system further comprises the application identifying at least one of a stock keeping unit associated with the compliance confirmation device on the computer network, a serial number associated with the compliance confirmation device on the computer network, and a device type associated with the compliance confirmation device on the computer network.
3. The method of any clause herein, responsive to determining the compliance confirmation device is not included in the computer network associated with the visualization system, blocking the data from being transmitted to the cloud-based data management system.
4. The method of any clause herein, wherein the compliance confirmation device is registered on the computer network after determining an agreement has been executed by an entity associated with the facility, wherein the agreement pertains to ownership of the data in the cloud-based data management system.
5. The method of any clause herein, wherein an account is created in the cloud-based data management system for the facility, one or more licenses are created for the account, and the method further comprises:
   receiving, by the application, at least one of the identification of the facility, a license identifier, and a license password.
6. The method of any clause herein, further comprising entering, via the application, a setup mode to configure the compliance confirmation device with the at least one of the identification of the facility, the license identifier, and the license password.
7. The method of any clause herein, further comprising displaying, by the application, a graphical element to indicate the compliance confirmation device is included in the computer network associated with the visualization system.
8. The method of any clause herein, further comprising, prior to transmitting the data to the cloud-based data management system, switching the application to operate in an internet-based communication protocol.
9. The method of any clause herein, wherein the visualization system is an arthroscopic tower and the compliance confirmation device is communicatively coupled to the arthroscopic tower.
10. The method of any clause herein, further comprising transmitting the license identifier and the license password to the cloud-based data management system to create a cloud session.
11. The method of any clause herein, further comprising receiving a serial number associated with a camera control unit of the visualization system.
12. The method of any clause herein, further comprising displaying, via the application, the identification of the facility associated with a license to upload the data to the cloud-based data management system.
13. The method of any clause herein, further comprises displaying, via the application, a list of one or more camera control units associated with one or more visualization systems that obtain data, wherein the list of the one or more camera control units are approved to upload data to the cloud-based data management system.
14. The method of any clause herein, wherein the cloud-based data management system comprises an exclusive mapping of the identification of the facility to an identification of the visualization system.
15. A system comprising:
a memory device storing instructions; and
a processing device communicatively coupled to the memory device, the processing device executes the instructions to:
receive, by an application implemented by the instructions, an input to initiate a patient session using a visualization system, wherein the processing device is communicatively coupled to the visualization system;
determine, by the application, whether a compliance confirmation device is included in a computer network associated with the visualization system, wherein the compliance confirmation device is associated with an identification of a facility at which the visualization system is used;
responsive to determining the compliance confirmation device is included in the computer network associated with the visualization system, receive the identification of the facility from the compliance confirmation device;
obtain, using the application, data during the patient session;
receive a request to transmit the data to a cloud-based data management system;
transmit the identification of the facility to the cloud-based data management system for verification; and
responsive to verifying the identification of the facility, transmit the data for storage at the cloud-based data management system.
16. The system of any clause herein, wherein the processing device is further to:
wherein determining, by the application, whether the compliance confirmation device is included in the computer network associated with the visualization system further comprises the application identifying at least one of a stock keeping unit associated with the compliance confirmation device on the computer network, a serial number associated with the compliance confirmation device on the computer network, and a device type associated with the compliance confirmation device on the computer network.
17. The system of any clause herein, wherein responsive to determining the compliance confirmation device is not included in the computer network associated with the visualization system, the processing device is to block the data from being transmitted to the cloud-based data management system.
18. The system of any clause herein, wherein the compliance confirmation device is registered on the computer network after determining an agreement pertaining to ownership of the data in the cloud-based data management system has been executed by an entity associated with the facility.
19. A tangible, non-transitory computer-readable medium storing instructions that, when executed, cause a processing device to:
receive, by an application implemented by the instructions, an input to initiate a patient session using a visualization system, wherein the processing device is communicatively coupled to the visualization system;
determine, by the application, whether a compliance confirmation device is included in a computer network associated with the visualization system, wherein the compliance confirmation device is associated with an identification of a facility at which the visualization system is used;
responsive to determining the compliance confirmation device is included in the computer network associated with the visualization system, receive the identification of the facility from the compliance confirmation device;
obtain, using the application, data during the patient session;
receive a request to transmit the data to a cloud-based data management system;
transmit the identification of the facility to the cloud-based data management system for verification; and
responsive to verifying the identification of the facility, transmit the data for storage at the cloud-based data management system.
20. The computer-readable medium of any clause herein, wherein determining, by the application, whether the compliance confirmation device is included in the computer network associated with the visualization system further comprises the application identifying at least one of a stock keeping unit associated with the compliance confirmation device on the computer network, a serial number associated with the compliance confirmation device on the computer network, and a device type associated with the compliance confirmation device on the computer network.

What is claimed is:

1. A method of operating a computing device, the method comprising:
receiving, by an application operating on the computing device, an input to initiate a patient session using a visualization system, wherein the computing device is communicatively coupled to the visualization system;
determining, by the application, whether a compliance confirmation device is included in a computer network associated with the visualization system, wherein the compliance confirmation device is associated with an identification of a facility at which the visualization system is used;
responsive to determining the compliance confirmation device is included in the computer network associated with the visualization system, receiving the identification of the facility from the compliance confirmation device;
obtaining, using the application, data during the patient session;
receiving a request to transmit the data to a cloud-based data management system;
transmitting the identification of the facility to the cloud-based data management system for verification; and
responsive to verifying the identification of the facility, transmitting the data for storage at the cloud-based data management system.

2. The method of claim 1, wherein determining, by the application, whether the compliance confirmation device is included in the computer network associated with the visualization system further comprises the application identifying at least one of a stock keeping unit associated with the compliance confirmation device on the computer network, a serial number associated with the compliance confirmation device on the computer network, and a device type associated with the compliance confirmation device on the computer network.

3. The method of claim 1, responsive to determining the compliance confirmation device is not included in the computer network associated with the visualization system, blocking the data from being transmitted to the cloud-based data management system.

4. The method of claim 1, wherein the compliance confirmation device is registered on the computer network after determining an agreement has been executed by an entity associated with the facility, wherein the agreement pertains to ownership of the data in the cloud-based data management system.

5. The method of claim 4, wherein an account is created in the cloud-based data management system for the facility, one or more licenses are created for the account, and the method further comprises:
receiving, by the application, at least one of the identification of the facility, a license identifier, and a license password.

6. The method of claim 5, further comprising entering, via the application, a setup mode to configure the compliance confirmation device with the at least one of the identification of the facility, the license identifier, the license password, a compliance contract identifier, a cloud-based data management system entity identifier, a user cloud account login, a user cloud account password, and a generic identifier that is alphanumeric.

7. The method of claim 1, further comprising displaying, by the application, a graphical element to indicate the compliance confirmation device is included in the computer network associated with the visualization system.

8. The method of claim 1, further comprising, prior to transmitting the data to the cloud-based data management system, switching the application to operate in an internet-based communication protocol.

9. The method of claim 1, wherein the visualization system is an arthroscopic tower and the compliance confirmation device is communicatively coupled to the arthroscopic tower.

10. The method of claim 5, further comprising transmitting the license identifier and the license password to the cloud-based data management system to create a cloud session.

11. The method of claim 1, further comprising receiving a serial number associated with a camera control unit of the visualization system.

12. The method of claim 1, further comprising displaying, via the application, the identification of the facility associated with a license to upload the data to the cloud-based data management system.

13. The method of claim 1, further comprises displaying, via the application, a list of one or more camera control units associated with one or more visualization systems that obtain data, wherein the list of the one or more camera control units are approved to upload data to the cloud-based data management system.

14. The method of claim 1, wherein the cloud-based data management system comprises an exclusive mapping of the identification of the facility to an identification of the visualization system.

15. A system comprising:
a memory device storing instructions; and
a processing device communicatively coupled to the memory device, the processing device executes the instructions to:
receive, by an application implemented by the instructions, an input to initiate a patient session using a visualization system, wherein the processing device is communicatively coupled to the visualization system;
determine, by the application, whether a compliance confirmation device is included in a computer network associated with the visualization system, wherein the compliance confirmation device is associated with an identification of a facility at which the visualization system is used;
responsive to determining the compliance confirmation device is included in the computer network associated with the visualization system, receive the identification of the facility from the compliance confirmation device;
obtain, using the application, data during the patient session;
receive a request to transmit the data to a cloud-based data management system;
transmit the identification of the facility to the cloud-based data management system for verification; and
responsive to verifying the identification of the facility, transmit the data for storage at the cloud-based data management system.

16. The system of claim 15, wherein the processing device is further to:
wherein determining, by the application, whether the compliance confirmation device is included in the computer network associated with the visualization system further comprises the application identifying at least one of a stock keeping unit associated with the compliance confirmation device on the computer network, a serial number associated with the compliance confirmation device on the computer network, and a device type associated with the compliance confirmation device on the computer network.

17. The system of claim 15, wherein responsive to determining the compliance confirmation device is not included in the computer network associated with the visualization system, the processing device is to block the data from being transmitted to the cloud-based data management system.

18. The system of claim 15, wherein the compliance confirmation device is registered on the computer network after determining an agreement pertaining to ownership of the data in the cloud-based data management system has been executed by an entity associated with the facility.

19. A tangible, non-transitory computer-readable medium storing instructions that, when executed, cause a processing device to:
receive, by an application implemented by the instructions, an input to initiate a patient session using a visualization system, wherein the processing device is communicatively coupled to the visualization system;
determine, by the application, whether a compliance confirmation device is included in a computer network associated with the visualization system, wherein the compliance confirmation device is associated with an identification of a facility at which the visualization system is used;
responsive to determining the compliance confirmation device is included in the computer network associated with the visualization system, receive the identification of the facility from the compliance confirmation device;
obtain, using the application, data during the patient session;
receive a request to transmit the data to a cloud-based data management system;
transmit the identification of the facility to the cloud-based data management system for verification; and
responsive to verifying the identification of the facility, transmit the data for storage at the cloud-based data management system.

20. The computer-readable medium of claim 19, wherein determining, by the application, whether the compliance confirmation device is included in the computer network associated with the visualization system further comprises the application identifying at least one of a stock keeping unit associated with the compliance confirmation device on the computer network, a serial number associated with the compliance confirmation device on the computer network, and a device type associated with the compliance confirmation device on the computer network.
